(19) United States
(12) Patent Application Publication
(10) Pub. No.: US 2009/0274750 A1
(43) Pub. Date: Nov. 5, 2009

(54) Therapeutic Treatment of Dermatologic Skin Disorders

(76) Inventor: Benjamin Johnson, Evergreen, CO (US)

Correspondence Address: GLENN L. WEBB;GLENN L. WEBB P.C., P.O BOX 3788, DURANGO, CO, 81302, US

(21) Appl. No.: 12/113,119

(22) Filed: Apr. 30, 2008

(51) Int. Cl. A61K 9/127; A61K 36/00; A61P 17/00

(52) U.S. Cl. 424/450; 424/750

(57) ABSTRACT

Willowherb derivatives are incorporated with liposomes in therapeutic compositions for topical application to prevent or alleviate the conditions and symptoms of cosmetic and dermatologic disorders. In particular, willowherb derivates are combined with liposomes to increase the penetration of the willowherb derivates into the skin, to provide hydration of the skin during treatment.

### FIELD OF THE INVENTION

This invention relates to the field of treatments for dermatologic and cosmetic skin disorders.

### BACKGROUND OF THE INVENTION

There are an extensive number of skin care products used for the treatment of skin disorders that may have resulted from aging, environment damage, disease or other factors. These disorders range from age spots, wrinkles, warts, acne, eczema, keratoses, psoriasis, xerosis, aging skin, biochemical disorders within the skin and many other disorders.

Overexposure to sunlight can especially damage the skin including not only sunburns, photosensitivity, premature aging but also including immunosuppression, and various types of skin cancers. The ultraviolet rays from the sunlight can also have a damaging effect on the collagen and elastin fibers causing premature aging and wrinkling of the overexposed skin.

One of the key factors in causing skin damage from overexposure to sunlight as well as in treating premature aging and wrinkling along with treating skin cancers and other ailments are from free radicals. Free radicals are highly reactive chemical species with an odd number of electrons that can react quickly with other compounds. This reaction can lead to cellular damage thus creating skin damage and ailments. Chemicals such as antioxidants help to protect the cells from the harmful effects of free radicals. Antioxidants are produced by the body as well as found in certain vitamins and minerals.

There are a number of skin care products that use a multitude of ingredients arranged in numerous formulations. These products have had varying amounts of successes in treating dermatologic and cosmetic skin disorders. Also, many individuals prefer to use natural products to treat skin disorder instead of chemical products.

One product that has shown promise in treating skin damage, particularly damage due to overexposure to ultraviolet rays as well as acne, and free radical damage are derivatives extracted from the willowherb. Epilobium angustifolium, commonly referred to as willowherb or fireweed has been long thought to have anti-inflammatory, anti-irritation properties and may have potential as an anti-oxidant. However, it has been difficult to apply it in deeply enough for its anti-oxidant qualities to be effective.

Thus a problem exists in providing topical skin care products containing willowherb derivatives and other therapeutic compounds that have increased efficacy in the treatment of many dermatologic and cosmetic skin disorders.

### SUMMARY OF THE INVENTION

The present invention solves these and other problems by providing willowherb derivatives with liposomes. The present invention may in various embodiments be used to increase the efficacy of the use of willowherb derivatives for therapeutically and cosmetically treating many skin disorders.

A preferred embodiment of the present invention increases the efficacy of a topical skin care product by increasing the penetration of willowherb derivatives into the skin. Compositions containing willowherb derivatives are coated or mixed with liposomal materials. The effect of willowherb derivatives on the skin occurs not only at the upper levels of the skin but also at the dermal level, so without adequate penetration, they have limited function in the skin. The liposomal willowherb derivatives compound has been shown to greatly increase the penetration of the willowherb derivatives which increases the efficacy of the product.

In a preferred embodiment of the present invention, the use of liposomal willowherb derivatives in a topical application also may be effective in the treatment of damaged skin by providing antioxidants and other medically therapeutic products into the skin cells. The use of willowherb derivates with liposomes increase the penetration of the to attack foreign bodies in the skin.

Another preferred embodiment of the present invention is useful in treating most common skin conditions, such as photodamage, acne, hyperpigmentation, rosacea and other conditions.

Another preferred embodiment of the present invention reduces the irritation to the skin. Compositions containing willowherb derivatives are coated or mixed with liposomal materials to reduce the irritation to the skin. Also the additional hydration of the skin using the liposomal formulation reduces the irritation as well.

These and other features of the present invention will be evident from the ensuing detailed description of preferred embodiments and from the claims.

### DETAILED DESCRIPTION OF PREFERRED EMBODIMENTS

The present invention provides products and methods for increasing the efficacy of treating skin disorders. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding of the claimed invention. It is to be expressly understood that other products and methods are contemplated for use with the present invention as well.

In accordance with the present invention, compounds derived from willowherb are incorporated with liposomes in therapeutic compositions for topical application to prevent or alleviate the conditions and symptoms of cosmetic and dermatologic disorders are described as follows. In particular, xanthones and other compounds derived from the willowherb are combined with liposomes to increase the penetration of those molecules into the skin, to provide hydration of the skin during treatment, and other embodiments and uses.

### Willowherb

One naturally occurring compound that is showing great promise is derived from Epilobium angustifolium, commonly referred to as willowherb or fireweed. The willowherb is widely grown naturally in both the temperate and subartic regions of both hemispheres and particularly in the northern United States and Canada. The willowherb is a mostly herbaceous plant with mostly opposite or whorled leaves.

The extracts from the willowherb have been well known to have soothing properties when applied to the skin. It has also been long thought to be helpful in reducing skin redness, anti-bacterial properties, anti-inflammatory properties and helpful in healing skin wounds and burns. More recently, it is believed to possible be a potent anti-oxidant.

The willowherb derivates are processed from the components of the willowherb including the leaves, stalks, flowers, juices and other naturally occurring components by known processing techniques including without limitation mixing, grinding, extraction by water, solvents or other techniques, cooking and any other known processing technique.

The compositions of the present invention may also include other cosmetic and/or pharmaceutical agents along with the derivatives of the willowherb to enhance their efficacy. Some examples of cosmetic and pharmaceutical agents are clotrimazole, miconazole, salicyclic acid, pramoxine, menthol, retinoic acid, hydrocortisone, hydrocortisone valerate, betamethasone valerate, betamethasone dipropionate, triamcinolone acetonide, fluocinonide, hydroquinone, clobetasol propionate, benzoyl peroxide, crotaminton, 5-fluorouracil, monobenzone, vitamin A palmitate, vitamin E acetate and vitamin C.

Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

Preparation of Exemplary Therapeutic Compositions

Accordingly, a preferred embodiment of the present invention provides cosmetic as well as medicinal compositions containing derivates from the willowherb coated in liposomal material which when topically administered will substantially improve and alleviate the symptoms of various cosmetic conditions or dermatologic disorders.

The willowherb derivatives of the instant invention may be formulated for topical application in aqueous or non-aqueous solution, gel, lotion, cream or ointment containing 0.1 to 80 percent and preferably from 0.5 to 20 percent by weight of the total composition. Other additives may be used to stabilize or otherwise provide functionality such as solvents, plasticizers, emulsify, stiffen, or other functions. Liposomal lecithin or a liposome substitute or other lipid preparations are added to the above solution with mixing until a uniform consistency is obtained.

To prepare a typical aqueous solution, the willowherb derivates are dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, the willowherb derivatives are dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved. When a combination composition is desired retinyl palmitate and/or hydroquinone, for example is added to the above non-aqueous solution. The preferred concentration of retinyl palmitate ranges from 1 to 5%. The concentration of hydroquinone may range from 1 to 5%, but the preferred concentration is 2% by weight of the total composition.

A typical cream or lotion containing willowherb derivates is prepared by first dissolving the willowherb derivatives in ethanol, acetone, propylene glycol or other solvent. The solution thus prepared is then admixed with commonly available oil-in-water emulsions. BHT or sodium metabisulfite may be added to such emulsions to stabilize the willowherb derivatives. Liposomes or liposome substitutes are added to this solution with mixing until a uniform consistency is achieved.

A typical gel composition is formulated by first dissolving the willowherb derivatives in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

Therapeutic Uses

The present invention may in various embodiments be used to increase the efficacy of the use of willowherb derivatives for therapeutically and cosmetically treating many skin disorders.

A preferred embodiment of the present invention increases the efficacy of a topical skin care product by increasing the penetration of willowherb derivatives into the skin. Compositions containing willowherb derivatives are coated or mixed with liposomal materials as described above. The liposomal willowherb derivatives compound has been shown to increase the penetration of the willowherb derivatives increases the efficacy of the willowherb derivatives product.

The willowherb derivatives then improves the health of damaged skin and in particular skin damaged by ultraviolet rays, suffering from certain types of skin cancer and other skin disorders. The willowherb derivates reduce the inflammation of the damaged skin, reduces the irritation of the skin and adds free radical scavenging activity. It is believed that this occurs at least in part by the antioxidant properties of the willowherb derivatives scavenging free radicals that may be causing the skin damage. This is critical in improving the look and health of skin that is damaged as part of the aging process and from external exposure to ultraviolet rays, smoking, free radicals and other factors. The preferred embodiment of the liposomal willowherb derivatives allow deeper penetration of this beneficial activity that has not been possible in prior topical compositions.

The effect of willowherb derivatives on the skin occurs at the dermal level, so without adequate penetration, they have limited function in the skin. The liposomal willowherb derivatives compound has been shown to greatly increase the penetration of the willowherb derivatives which increases the efficacy of the product.

Another preferred embodiment of the present invention is useful in treating scar tissue that results from most common skin conditions, such as photodamage, acne, hyperpigmentation, rosacea and other conditions.

The use of liposomal willowherb derivatives also increases the efficacy of the willowherb derivatives by increasing the delivery of the willowherb derivatives that would normally irritate the skin layers. The derivatives are delivered deeper into the skin layers without irritating the upper skin layers which normally occurs. This provides the benefits from the antioxidant characteristics of the willowherb derivatives without the skin irritation that prevents it from otherwise being delivered deeper into the skin.

Another preferred embodiment of the present invention increased the hydration of the skin from using willowherb derivatives. Compositions containing willowherb derivatives are coated or mixed with liposomal materials as described above. Liposomes have a natural affinity for water which assists in increasing the moisture in the skin during topical application of the liposomal willowherb derivatives composition.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

What is claimed is:

1. A method for increasing the transdermal penetration of willowherb derivatives compounds from a topical skin care application, said method comprising the steps of:
providing a willowherb derivate compound in the skin care application; and
providing said willowherb derivate compound within a liposome compound to increase the transdermal penetration of said compound.

2. The method of claim 1 for increasing the transdermal penetration of willowherb derivates from a topical skin care application of claim 1 wherein step of providing said compound includes:
providing said compound in the range of between 0.1 percent to 20 percent by weight.

3. The method of claim 1 for increasing the transdermal penetration of willowherb derivate from a topical skin care application of claim 1 wherein step of providing said compound includes:
providing lipid compounds having enhanced penetration properties.

4. The method of claim 1 for increasing the transdermal penetration of willowherb derivate from a topical skin care application of claim 1 wherein step of providing said compound includes:
providing said liposome compound in the range of 0.1 percent to 20 percent by weight.

5. The method of claim 1 for increasing the transdermal penetration of willowherb derivate from a topical skin care application of claim 1 wherein step of providing said compound includes:
providing a lipid compound having a high affinity for water.

6. A topical composition for therapeutic treatment of skin, said composition comprising:
a liposomal compound; and
a willowherb derivate compound contained within said liposomal compound.

7. The topical composition of claim 6 wherein said composition includes:
willowherb derivate compound in a range of between 0.1 and 20 percent by weight.

8. The topical composition of claim 6 wherein said composition includes:
said liposomal compound has a high affinity for water.

9. A method for treating damaged skin, said method comprising:
providing a liposomal compound;
providing a willowherb derivate compound contained within said liposomal compound;
applying said willowherb derivate compound contained within said liposomal compound to the damaged skin.

10. The method for treating damaged skin of claim 9 wherein step of providing said willowherb derivate compound includes:
providing said willowherb derivate compound in the range of between 0.1 percent to 20 percent by weight.
